(19) United States
(12) Patent Application Publication
(10) Pub. No.: US 2022/0296188 A1
(43) Pub. Date: Sep. 22, 2022

(54) MOBILE X-RAY DEVICE AND METHOD FOR OPERATING A MOBILE X-RAY DEVICE

(71) Applicant: Siemens Healthcare GmbH, Erlangen (DE)

(72) Inventors: Silvia Meyer, Erlangen (DE); Markus Kowarschik, Nürnberg (DE)

(21) Appl. No.: 17/697,903

(22) Filed: Mar. 17, 2022

(30) Foreign Application Priority Data
Mar. 18, 2021 (DE) 10 2021 202 662.1

(51) Int. Cl. A61B 6/00; A61B 6/08

(52) U.S. Cl. CPC A61B 6/4405; A61B 6/54; A61B 6/463; A61B 6/4441; A61B 6/4078; A61B 6/4085; A61B 6/08

(57) ABSTRACT

A mobile X-ray device with an adjustable recording system that is arranged on a device trolley is provided. The adjustable recording system has an X-ray source and an X-ray detector for recording X-ray images of an object. The mobile X-ray device includes a system controller for actuating the X-ray device, a calculation unit for real-time determination of a scattered radiation distribution from an X-ray radiation generated by the X-ray source in at least parts of the surrounding area of the X-ray device, and a display unit that is arranged on the device trolley and/or the recording system. The display unit is configured for display, taking place substantially in real time, of at least one item of information dependent upon the determined scattered radiation distribution in at least one part of the surroundings of the mobile X-ray device.

This application claims the benefit of German Patent Application No. DE 10 2021 202 662.1, filed on Mar. 18, 2021, which is hereby incorporated by reference in its entirety.

### BACKGROUND

The present embodiments relate to a mobile X-ray device with an adjustable recording system that is arranged on a device trolley, as well as a method for operating a mobile X-ray device.

During X-ray imaging, it is known in principle in the prior art that scattered radiation effects occur due to interaction of the X-ray radiation used (e.g., with the patient to be recorded himself). In this context, two essential kinds of scattered radiation are known: forward-scattered X-ray radiation may bring uneven exposure and contrast degradation in recorded X-ray images; according to investigations, back-scattered radiation constitutes 30 to 60% of the overall main dose of the patient and also endangers persons located in the surrounding area of the X-ray device, such as doctors and operators, for example. In permanently installed X-ray devices, it is therefore often the case that the operator will leave the surrounding area of the X-ray device and only then is the X-ray radiation triggered remotely.

In mobile X-ray devices, however, this is often not possible. Mobile X-ray devices also is to be used in non-shielded regions; in many cases, a medical intervention is to be performed at the same time as the X-ray recording or with X-ray monitoring. For these reasons, for example, the doctor and/or the operator remain in the immediate surrounding area of the radiation source of the X-ray device during the triggering of the radiation. In this case, it is often unclear where the safest position with the lowest radiation exposure (e.g., in relation to the scattered radiation) is located for the operator.

In devices of this kind, it is known that the operator is prompted by the X-ray device to wear a lead apron and, if necessary, is informed to position himself outside of the X-ray radiation. This is difficult if it is not clear where precisely scattered radiation is located, and how much.

It is known to calculate a scattered radiation distribution in space using elaborate simulations (e.g., Monte Carlo simulations). This, however, requires a large amount of computing effort and lasts hours or days. From patent application DE 10 2019 215 242.2, which is not a prior publication, a method is known in which a scattered radiation distribution in space is ascertained in real time with the aid of trained machine learning functions.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a mobile X-ray device that offers a simple option for largely avoiding scattered radiation to an operator located near the mobile X-ray device is provided. As another example, a method for operating a mobile X-ray device of this kind is provided.

A mobile X-ray device according to an embodiment with an adjustable recording system that is arranged on a device trolley is provided. The adjustable recording system has an X-ray source and an X-ray detector for recording X-ray images of an object. The mobile X-ray device also includes a system controller for actuating the X-ray device, a calculation unit for real-time determination of a scattered radiation distribution from an X-ray radiation generated by the X-ray source in at least parts of the surrounding area of the X-ray device (e.g., as a function of at least one influencing variable that is variable over time), and a display unit that is arranged on the device trolley and/or the recording system. The display is configured for the display, taking place substantially in real time, of at least one item of information dependent upon the determined scattered radiation distribution (e.g., distribution of the scattered radiation dose in space) in at least one part of the surroundings of the mobile X-ray device.

Using the mobile X-ray device according to the present embodiments, it is possible for supporting operators or doctors to identify, substantially in real time and therefore in a particularly rapid manner, which positions are hazardous with regard to scattered radiation and which are relatively safe. As a result, the operators or doctors are able to respond immediately and remove themselves from the hazardous regions and/or position themselves at the safer positions and therefore minimize their risk of an impairment to their health. The mobile X-ray device according to the present embodiments therefore has a high level of user friendliness.

According to one embodiment, the display unit is embodied for the visual display (e.g., projection) of an item of information dependent upon the determined scattered radiation distribution in at least one part of the surroundings of the mobile X-ray device. For example, the display unit is embodied for the visual (e.g., color-based) marking of at least one part of an area surrounding the X-ray device (e.g., the floor, underfloor or ceiling) with regard to the scattered radiation distribution determined for a volume arranged above and/or below the area. In this manner, the operator is able to identify, on a color projection, for example, in a rapid and unambiguous manner, that he is located in a hazardous region and is able to proceed to another region (e.g., marked by another color). A projection, for example, of the scattered radiation dose occurring in the spatial region above the floor, onto the floor located below (or another surface), or of the scattered radiation dose occurring below onto the ceiling is a particularly simple and low-outlay option in this context of making the hazardous regions and safe regions visible. Overall, for example, a red-yellow-green colored marking may be used, as this is generally understandable.

According to a further embodiment, for this purpose, the display unit may be formed of at least one color projector (e.g., adjustable color projector) that is mounted on the recording system. A plurality or a large number of small projectors may also be used, which, for example, due to their distribution at various positions and/or their adjustable arrangement on the X-ray device or the recording system are able to project onto any region of the floor. As an alternative, it is also possible to project onto the ceiling (e.g., the scattered radiation distribution occurring in the spatial region below the ceiling may be projected onto the ceiling located thereabove).

According to a further embodiment, the X-ray device is formed by a mobile computed tomography unit with a rotatable gantry and an X-ray source that generates a fan beam or cone beam. Such a device may also be embodied for recording volume image data. A device that may be moved and, for example, may be used for CT recording of the head is known, for example, by the name SOMATOM On.site® of the company Siemens Healthineers.

According to a further embodiment, the X-ray device is formed by a mobile C-arm X-ray device with an adjustable C-arm and an X-ray source that generates a cone beam. Such a device may also be embodied for recording volume image data. Movable C-arm X-ray devices that are arranged on a device trolley that have C-arms that may be adjusted for 3D recording, for example, are known (e.g., the device cios Spin® of the company Siemens Healthineers).

According to a further embodiment, the influencing variable that is variable over time is formed by a spatial position of the X-ray device and/or a recording parameter (e.g., X-ray voltage or current) and/or an item of patient information and/or a position of the recording system. In this context, the spatial position of the X-ray device is variable, for example, by the X-ray device being moved automatically or manually. The position of the recording system is variable, for example, by the recording system (e.g., a C-arm) being adjusted (e.g., rotated) and/or moved over an examination object for a large number of successive 2D projections (e.g., which projections may then be reconstructed to form a volume image for example) or, in the case of a CT gantry, by the recording system being displaced along the examination object during the recording. The recording parameters may be variable by a plurality of recordings taking place successively with different recording parameters (e.g., X-ray voltage or current, zoom, filter settings, etc.). The patient form currently being irradiated may change in that the recording system is moved relative to the examination object during the recording (e.g., different regions of the examination object are exposed in succession). For all these influencing variables that are variable (e.g., over time), it is a great advantage if a real-time update of the determination of the scattered radiation distribution is created and displayed and therefore each modification of the scattered radiation distribution may be immediately clearly identified by a user. In this manner, the user is able to respond rapidly, proceed from a possible hazardous region to a non-hazardous region in a timely manner, and therefore minimize their health risk.

According to a further embodiment, the X-ray device has a sensor (e.g., a camera) for determining position information of at least one person located in the surrounding area. A plurality of cameras may also be provided in order to track multiple people or determine the positions thereof, for example. A touch sensor may also be present, for example, on a handle element attached to the X-ray device. The handle element is embodied to be touched by a person. If the person touches the handle element, then the touch sensor is able to detect the position of the person.

According to a further embodiment, the X-ray device has an evaluation unit for evaluating the position information of the person with regard to the determined scattered radiation distribution. Thus, the evaluation unit is able to compare the position information of the person(s) ascertained by the sensor with the current scattered radiation distribution, in order to ascertain whether or not the person(s) is/are located in a hazardous or non-hazardous region. It is also possible to determine the level of the scattered radiation to which the person(s) is/are exposed.

According to a further embodiment, the X-ray device has an acoustic output unit for outputting acoustic warning signals if the result of the evaluation of the evaluation unit is an exceeding of a threshold value of the determined scattered radiation. If a person is therefore located in a hazardous region of the scattered radiation, then, for example, an acoustic warning signal is output and/or the person is prompted to change position. It is also possible to output a confirmation signal if the person is located at a non-hazardous position.

According to a further embodiment, the calculation unit is embodied for real-time determination of the scattered radiation distribution with the use of a pretrained machine algorithm (e.g., learning algorithm). The machine algorithm may have been trained, for example, through the use of previously known scattered radiation distributions in space. The scattered radiation distributions come, for example, from real measurements or calculated simulations (e.g., Monte Carlo simulations). For example, updated values of the influencing variable that may be varied are then transmitted to the algorithm as input data in real time, and an updated scattered radiation distribution is immediately ascertained therefrom. For example, from patent application DE 10 2019 215 242.2, which is not a prior publication, a method is known in which a scattered radiation distribution in space is ascertained in real time with the aid of trained machine learning algorithms.

According to a further embodiment, the calculation unit is embodied for real-time determination of the scattered radiation distribution with the use of at least one further variable or non-variable influencing variable. It is thus possible to use a plurality of influencing variables that are variable over time as input. Additionally, it is also possible for influencing variables that are not variable over the duration of the method, such as fixed recording parameters or the position of shielding elements, for example, to be used in the calculations.

According to the present embodiments, a method for operating a mobile X-ray device described above with the following acts is additionally provided: provision of a pretrained machine algorithm for determining a scattered radiation distribution generated by a (back)scattering of the X-ray beam generated by the X-ray source in the surrounding area of the X-ray device as a function of at least one influencing variable that may be varied over time; activation of the X-ray device with regard to the emission of an X-ray beam; provision of current values of the at least one influencing variable that may be varied; real-time determination of the scattered radiation distribution by the pretrained machine algorithm with the use of the current values of the at least one influencing variable that may be varied; substantially real-time display of at least one item of information dependent upon the determined scattered radiation distribution in at least one part of the surroundings of the mobile X-ray device; and repetition of the provision of the current values of the at least one influencing variable, the real-time determination of the scattered radiation distribution, and the substantial real-time display in a continuous, triggered, or temporally spaced manner, as long as the X-ray beam is activated.

### BRIEF DESCRIPTION OF THE DRAWINGS

FIG. 1 shows a view of a mobile X-ray device in the form of a mobile computed tomography unit according to an embodiment;

FIG. 2 shows a further view of the mobile computed tomography unit of FIG. 1;

FIG. 3 shows a view of a further mobile X-ray device in the form of a mobile C-arm X-ray device according to an embodiment;

FIG. 4 shows a view of an exemplary scattered radiation distribution display projected by a mobile X-ray device; and

FIG. 5 shows a sequence of the acts of a method for operating a mobile X-ray device according to an embodiment.

### DETAILED DESCRIPTION

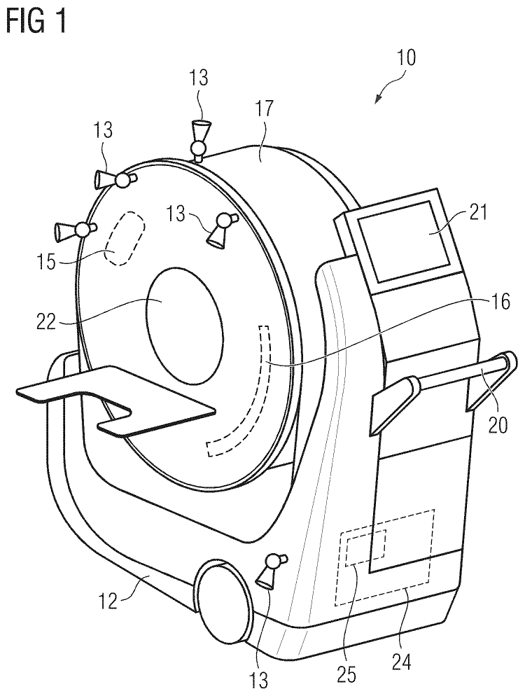

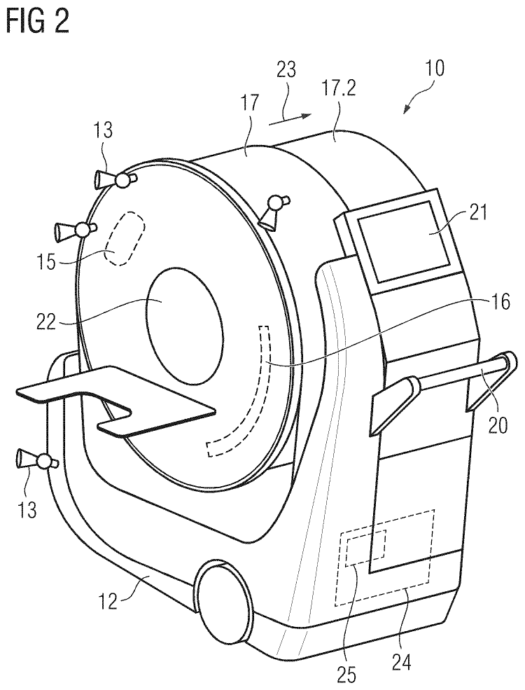

FIG. 1 shows one embodiment of a mobile X-ray device in the form of a mobile computed tomography unit 10 with a gantry 17 arranged on a device trolley 12. Located below a stationary housing (therefore not visible from the outside) is a rotatable recording system with an X-ray source 15 and an X-ray detector 16 (both shown with dashed lines). The rotatable recording system may be rotated about an opening 22 for positioning of an examination object. The X-ray source 15 is embodied to emit a fan beam; the X-ray detector 16 may involve a line detector, for example. For a cone beam, an area detector may alternatively also be present. For scanning the examination object with the rotatable recording system, the housing is embodied such that the housing may be extended telescopically in the arrow direction 23 by a second housing part 17.2 with the recording system being pushed out (see FIG. 2). In this context, the rotatable recording system sweeps over the examination object in arrow direction 23 at the same time as the rotation and records image data that may then be reconstructed to form a volume image of the examination object.

The mobile computed tomography unit 10 has a system controller 24 (e.g., a control computer) for actuating the mobile X-ray device. Additionally, a calculation unit 25 is provided for real-time determination of a scattered radiation distribution in the surrounding area of the X-ray device as a function of at least one influencing variable that, for example, is variable over time. The calculation unit 25 may, for example, be integrated into the system controller 24 or have a communication connection with the system controller 24. The calculation unit 25 may, for example, have a trained machine learning algorithm that, in a very short time (e.g., almost real time), is able to calculate a scattered radiation distribution in the surrounding area of the X-ray device. In this context, the calculation unit 25 uses current values of the at least one influencing variable that may be varied over time as input. The current values of the influencing variable that may be varied over time are transmitted to the calculation unit 25, for example, by the system controller 24. A maximum surrounding area of the X-ray device to be covered is stipulated before the method (e.g., automatically or manually by a user). Before or during the method, it is possible to set or select a limiting of the surrounding area for which the scattered radiation distribution is determined.

An influencing variable that is variable (e.g., over time) may, for example, be formed by a spatial position of the X-ray device and/or a recording parameter (e.g., X-ray voltage or current) and/or an item of patient information and/or a position of the recording system. In this context, the spatial position of the X-ray device may be varied, for example, by the X-ray device being moved automatically or manually. The position of the recording system may be varied, for example, by the recording system (e.g., a C-arm) being adjusted for a large number of successive 2D projections (e.g., that may then be reconstructed to form a volume image for example) or, in the case of a CT gantry, by the recording system being displaced over the examination object during the recording. The recording parameters may be variable by a plurality of recordings following one another with different recording parameters. The patient form currently being irradiated may change in that the recording system is moved relative to the examination object during the recording (e.g., different regions of the examination object are exposed in succession). Patient movement (e.g., caused by respiration or heartbeat) may also lead to temporal invariance. Here, for example, additional sensors may be provided in order to capture any patient movements and take the patient movements into consideration accordingly.

In general, the size of the safest region with regard to the scattered radiation is dependent upon the nature of the irradiated object.

The machine learning algorithm has been trained in a known manner before the starting of the method (e.g., one time or repeatedly). It is possible, for example, to use a large number of scattered radiation distributions simulated with the use of different values of the variable influencing variable. Scattered radiation distributions of this kind have been created in an elaborate manner (e.g., by Monte Carlo simulation). From patent application DE 10 2019 215 242.2, which is not a prior publication, a method is known in which a scattered radiation distribution in space is ascertained in real time with the aid of trained machine learning functions. As an alternative or in addition, it is also possible to use real measured scattered radiation distributions for the training. The artificial intelligence algorithm trained by machine learning is used in order to establish relationships between influencing variables that are variable and the scattered radiation, as at least partially also given by the X-ray image used as input data. In this context, for example, physical concepts and prior knowledge may be combined as limiting paths with a data-driven (e.g., convolutional) encoder-decoder model as residual path.

Additionally, the mobile computed tomography unit 10 has a plurality of projectors 13 that display at least one item of information regarding the scattered radiation distribution calculated by the calculation unit 25 in the surrounding area. Thus, the projectors 13 may, for example, project areas of color onto the floor or the ceiling, where the areas of color display, for example, the scattered radiation dose determined for a volume arranged above and/or below the area. The colors are selected, for example, as a function of threshold values for the scattered radiation dose, so that as of a predetermined value of the scattered radiation dose, an associated color is used.

It is also possible for a single, large projector to be provided. As an alternative or in addition, it is also possible for other display apparatuses to be arranged on the mobile computed tomography unit 10. It is also possible for colored holograms to be projected by one or more 3D projectors. The projector or the projectors are arranged on the mobile X-ray device in the form of a mobile computed tomography unit 10 itself (e.g., on the gantry 17 or the device trolley 12). If a large number of projectors 13 are present, these may be arranged at various positions on the mobile computed tomography unit 10 (e.g., evenly distributed over the mobile computed tomography unit 10) in order to be able to mark, if necessary, all the surrounding areas on the floor, on an underfloor, or on the ceiling with color.

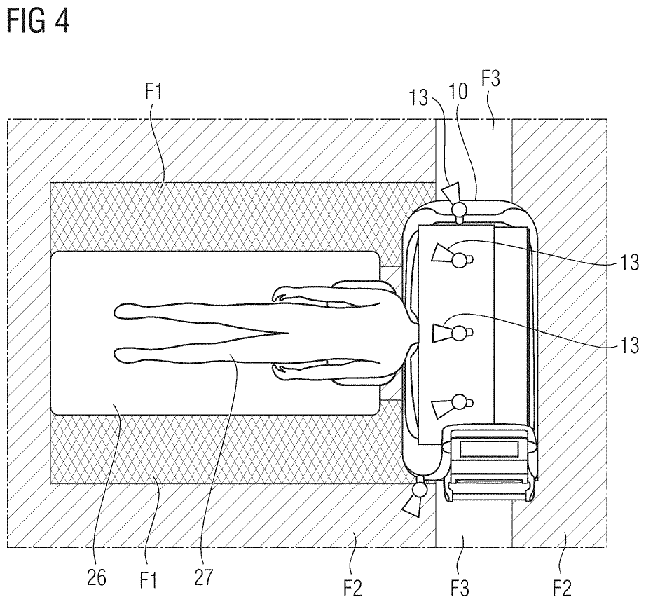

FIG. 4 shows a display of areas of color F1 to F3 (not necessarily connected) on the floor surrounding the mobile computed tomography unit 10 and the patient 27 positioned on the patient table 26 by one or more projectors 13. The first area F1 is generated, for example, in a first color (e.g., red). For the volume arranged above the first area, the determined scattered radiation dose (e.g., scattered radiation per area unit) exceeds a previously stipulated second threshold value S2. The second area F2 is illuminated in a second color (e.g., yellow), as here the determined scattered radiation dose (e.g., scattered radiation per area unit) lies below the second threshold value S2 but above a first threshold value S1 for the volume arranged above the second area. The third area is illuminated in a third color (e.g., green), as here the scattered radiation dose (e.g., scattered radiation per area unit) determined for the volume arranged above the third area lies below the first threshold value S1. The following applies: S1<S2. Color transitions may also be projected. As an alternative, it is also possible to only illuminate with color the regions that represent a particular hazard (e.g., in red, for a scattered radiation dose above a threshold value) or only the regions that are particularly safe (e.g., in green, such as for a scattered radiation dose below a threshold value). It is also possible for other surfaces (e.g., the patient table 26 or the patient 27) to be illuminated with color.

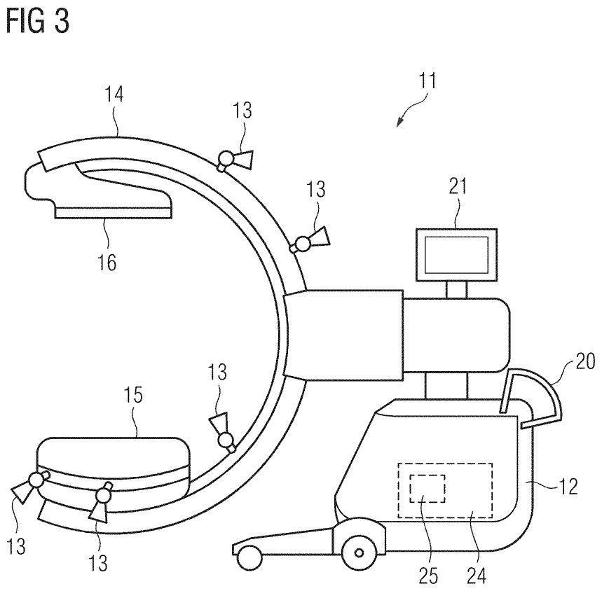

FIG. 3 shows one embodiment of a mobile X-ray device in the form of a mobile C-arm X-ray device 11 with an adjustable C-arm 14 and an X-ray source 15 that generates a cone beam as well as a flat X-ray detector 16. The projector(s) 13 is/are, for example, arranged on the C-arm 14 (e.g., on the X-ray source 15 and/or the X-ray detector 16) and/or on the device trolley 12. The C-arm X-ray device 11 also has a system controller 24 and a calculation unit 25 and functions in a similar manner to the mobile computed tomography unit 10.

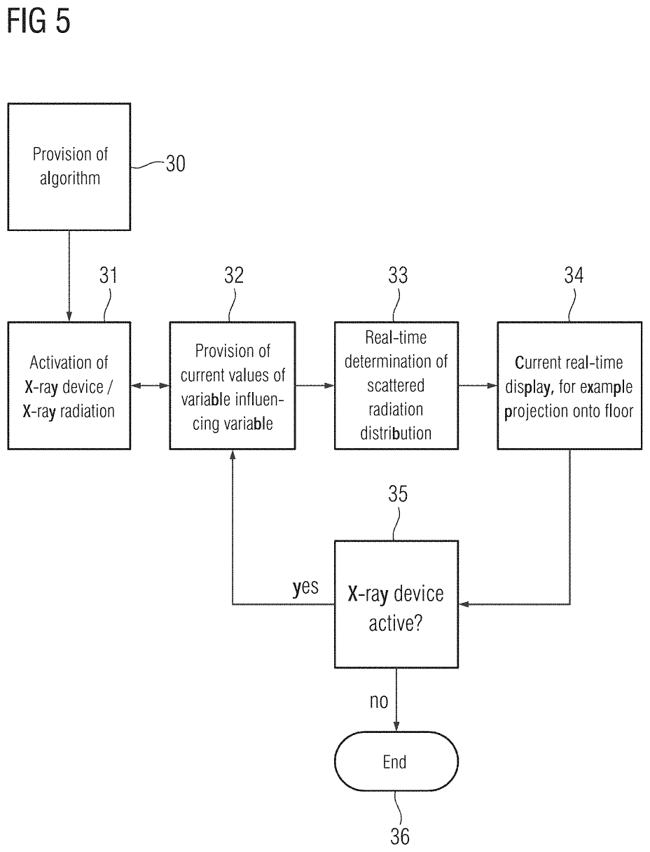

FIG. 5 shows a sequence of acts of one embodiment of a method for operating a mobile X-ray device (e.g., mobile computed tomography unit 10 or mobile C-arm X-ray device 11) described above. The method may, for example, be started and performed automatically or semi-automatically (e.g., autonomously or after receiving a user input). In a first act 30, a pretrained machine learning algorithm for determining a scattered radiation distribution generated by a backscattering of the X-ray beam generated by the X-ray source in the surrounding area of the X-ray device as a function of at least one influencing variable that may be varied is provided (e.g., by being loaded onto the calculation unit 25). The machine learning algorithm, as described above, for example, has been pretrained with training data (e.g., simulations or measurements of scattered radiation distributions) and is therefore available for real-time calculation of the scattered radiation distribution.

In a second act 31, the X-ray device is activated with regard to the emission of an X-ray beam. This may be performed, for example, automatically by a recording program or semi-automatically (e.g., after receiving a user input), for example. An examination object (e.g., an organ or body part of a patient) is arranged in the beam path of the X-ray beam. In the case of a mobile computed tomography unit 10, a head of a patient, for example, is therefore arranged such that the fan beam or cone beam irradiates a region of the head, and rotates around the head while doing so. In the case of a mobile C-arm X-ray device 11, for example, a region of a femur of a patient may be irradiated by a cone beam.

At the same time or almost at the same time as the second act 31, at least one current value of the at least one influencing variable that may be varied (see above) is provided (e.g., the current scanned patient form or the current position of the recording system or recording parameter (tube voltage)). The provision may be performed, for example, such that the current value of the at least one influencing variable that may be varied is, for example, ascertained or queried or retrieved by the system controller 24 and forwarded to the calculation unit 25.

Almost at the same time or after a very brief time, in a fourth act 33, the scattered radiation distribution is then determined in real time by the calculation unit and the pretrained machine algorithm with the use of the current value(s) of the at least one influencing variable that may be varied, and the scattered radiation distribution is immediately displayed in a fifth act 34. The display takes place, as described above, for example, by one or more projectors 13 (e.g., by projecting areas of color onto the floor or the ceiling in the surrounding area of the X-ray device). In this context, for example, it is possible to mark all the surroundings within a predetermined radius, or only hazardous or non-hazardous regions.

The third act 32, the fourth act 33, and the fifth act 34 are repeated until the X-ray beam is deactivated (e.g., automatically or manually). In this context, the repetition may take place in a continuous manner, a triggered manner (e.g., by capturing a change in the influencing variable that may be varied), or at preset temporal intervals. For this purpose, for example, it is possible for an automatic query 35 regarding the activation of the X-ray beam to take place. If the X-ray beam is deactivated (e.g., if an X-ray recording has concluded or an input for deactivation is received and implemented), then the ending 36 of the method takes place.

In addition to the visual display, it is also possible for an acoustic output or announcement that indicates safe or unsafe regions to be provided. This may be performed, for example, by an acoustic output unit (e.g., a microphone) that is arranged on the X-ray device. Thus, for example, the user may be acoustically notified of the region in which the user is in the safest position with regard to the scattered radiation distribution. This may also be performed in conjunction with one or more sensors for position detection. One or more sensors of this kind for position detection (e.g., cameras or touch sensors) may be arranged on the X-ray device. Using the sensors, the position of one or more persons is captured, forwarded to an evaluation unit, and evaluated with regard to the scattered radiation distribution. Subsequently, for example, a warning signal or an announcement is output if the result of the evaluation with regard to the position of the person is an exceeding of a threshold value of the determined scattered radiation. It is also possible to output a confirmation if the person is located at a safe position.

A touch sensor may, for example, be arranged on a handle element 20 (e.g., the product maneuvering handle (drive handle)).

In a further embodiment, the calculation unit 25 is embodied for real-time determination of the scattered radiation distribution with the use of at least one further variable or non-variable influencing variable. It is thus possible to use a plurality of influencing variables that are variable over time as input. Additionally, it is also possible for fixed influencing variables, such as recording parameters that are fixed during the method or the form and position of shielding elements, for example, to be used in the calculations. Such shielding elements may, for example, be fastened to the X-ray device or freely positioned in the surroundings of the X-ray device. To determine the spatial relationship between X-ray device and shielding element, it is possible, for example, to use a tracking system or a position detection system for tracking or position detection of the shielding element (e.g., visually, electromagnetically, etc.).

For a particularly high level of user-friendliness and hazard minimization, a mobile X-ray device with an adjustable recording system that is arranged on a device trolley is provided. The recording system has an X-ray source and an X-ray detector for recording X-ray images of an object. The mobile X-ray device includes a system controller for actuating the X-ray device, a calculation unit for real-time determination of a scattered radiation distribution from an X-ray radiation generated by the X-ray source in at least parts of the surrounding area of the X-ray device (e.g., as a function of at least one influencing variable that is variable over time), and a display unit that is arranged on the device trolley and/or the recording system. The display unit is configured for the display, taking place substantially in real time, of at least one item of information dependent upon the determined scattered radiation distribution in at least one part of the surroundings of the mobile X-ray device.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A mobile X-ray device comprising:
a device trolley;
an adjustable recording system that is arranged on the device trolley, the adjustable recording system comprising an X-ray source and an X-ray detector for recording X-ray images of an object;
a system controller configured to actuate the mobile X-ray device;
a calculation unit configured for real-time determination of a scattered radiation distribution from an X-ray radiation generated by the X-ray source in at least parts of a surrounding area of the mobile X-ray device; and
a display unit that is arranged on the device trolley, the recording system, or the device trolley and the recording system, the display unit being configured to display, substantially in real time, at least one item of information dependent upon the determined scattered radiation distribution in at least one part of surroundings of the mobile X-ray device.

2. The mobile X-ray device of claim 1, wherein the calculation unit is further configured to determine the scattered radiation distribution from the X-ray radiation generated by the X-ray source in at least the parts of the surrounding area of the mobile X-ray device as a function of at least one influencing variable that is variable over time.

3. The mobile X-ray device of claim 1, wherein the display unit is configured for visual display of an item of information dependent upon the determined scattered radiation distribution in at least one part of the surroundings of the mobile X-ray device.

4. The mobile X-ray device of claim 3, wherein the display unit is configured for projection of the item of information dependent upon the determined scattered radiation distribution in the at least one part of the surroundings of the mobile X-ray device.

5. The mobile X-ray device of claim 1, wherein the display unit is configured for visual marking of at least one part of an area surrounding the mobile X-ray device with regard to the scattered radiation distribution determined for a volume arranged above the area.

6. The mobile X-ray device of claim 5, wherein the display unit is configured for color-based visual marking of the at least one part of the area surrounding the mobile X-ray device with regard to the scattered radiation distribution determined for the volume arranged above the area.

7. The mobile X-ray device of claim 1, wherein the display unit comprises at least one color projector that is mounted on the adjustable recording system.

8. The mobile X-ray device of claim 1, wherein the mobile X-ray device is formed by a mobile computed tomography unit with a rotatable gantry, and
wherein the X-ray source is configured to generate a fan beam or a cone beam.

9. The mobile X-ray device of claim 1, wherein the X-ray device is formed by a mobile C-arm X-ray device with an adjustable C-arm, and
wherein the X-ray source is configured to generate a cone beam.

10. The mobile X-ray device of claim 2, wherein the influencing variable that is variable over time is formed by a spatial position of the mobile X-ray device, a recording parameter, an item of patient information, a position of the recording system, or any combination thereof.

11. The mobile X-ray device of claim 1, further comprising a sensor configured to determine position information of at least one person located in the surrounding area.

12. The mobile X-ray device of claim 11, wherein the sensor is a camera.

13. The mobile X-ray device of claim 11, further comprising an evaluation unit configured to evaluate the position information of the person with regard to the determined scattered radiation distribution.

14. The mobile X-ray device of claim 13, further comprising an acoustic output unit configured to output acoustic warning signals when a result of the evaluation is an exceeding of a threshold value of the determined scattered radiation.

15. The mobile X-ray device of claim 1, wherein the adjustable recording system is configured to record volume image data.

16. The mobile X-ray device of claim 1, wherein the calculation unit is configured for real-time determination of the scattered radiation distribution with the use of a pretrained machine algorithm.

17. The mobile X-ray device of claim 2, wherein the calculation unit is configured for real-time determination of the scattered radiation distribution with the use of at least one further variable or non-variable influencing variable.

18. A method for operating a mobile X-ray device, the method comprising:
providing a pretrained machine learning algorithm for determining a scattered radiation distribution generated by a scattering of an X-ray beam generated by an X-ray source of the mobile X-ray device in a surrounding area of the mobile X-ray device as a function of at least one influencing variable that is variable over time during the duration of the method;
activating the mobile X-ray device with regard to emission of an X-ray beam;
providing current values of the at least one variable influencing variable;
real-time determining of the scattered radiation distribution by the pretrained machine algorithm with the use of the current values of the at least one variable influencing variable;
substantially real-time displaying of at least one item of information dependent upon the determined scattered radiation distribution in at least one part of the surroundings of the mobile X-ray device; and
repeating the providing of the current values of the at least one variable influencing variable, the real-time determining of the scattered radiation distribution, and the substantially real-time displaying in a continuous, triggered, or temporally spaced manner as long as the X-ray beam is activated.

19. The method of claim 18, wherein the variable influencing variable is formed by a spatial position of the mobile X-ray device, a recording parameter, an item of patient information, a position of the recording system, or any combination thereof.

20. The method of claim 18, wherein at least one further variable or non-variable influencing variable is used.
